(19) United States
(12) Patent Application Publication
(10) Pub. No.: US 2024/0002763 A1
(43) Pub. Date: Jan. 4, 2024

(54) MICROFLUIDIC CELL CULTURE DEVICE

(71) Applicant: MICRONIT HOLDING B.V., Enschede (NL)

(72) Inventors: Sandro MEUCCI, Enschede (NL); Bianka FABINYI, Enschede (NL); Jasper Hendrik TEN NAPEL, Enschede (NL); Marko Theodoor BLOM, Delden (NL)

(73) Assignee: MICRONIT HOLDING B.V., Enschede (NL)

(21) Appl. No.: 18/029,839

(22) PCT Filed: Oct. 6, 2021
(86) PCT No.: PCT/NL2021/050606

§ 371 Date: Mar. 31, 2023

(30) Foreign Application Priority Data
Oct. 6, 2020 (NL) 2026633

(51) Int. Cl. C12M 3/06; C12M 1/00

(52) U.S. Cl. CPC C12M 23/16; C12M 29/10

(57) ABSTRACT

A microfluidic cell culture device includes a body having a fluid system including a first fluid chamber, a second fluid chamber, a cell culture chamber and a flow channel for allowing a flow of fluid from the first chamber, via the cell culture chamber, towards the said second chamber upon coupling of a pressure pump system to said fluid system. The fluid system also includes a return channel formed in the body allowing a flow of fluid from the second chamber to the first chamber.

The present invention relates to a microfluidic cell culture device for circulation of fluids, in particular for circulation of liquids. The present invention further relates to a method for culturing cells in a cell culture chamber.

In a cell culture, cells are grown under controlled conditions, for instance for the purpose of supporting medical research. Traditional in vitro systems were developed to be cost-efficient and to allow scalability in manufacturing the in vitro systems. These in vitro systems are therefore relatively simplistic. In contrast, the physiological systems which the in vitro systems are to simulate are complex. In physiological systems, cells are dynamically stimulated by various signals, for instance chemical or mechanical. Such chemical and mechanical signals are usually absent from traditional in vitro systems. Their lack of biomimicry, i.e. the ability to stimulate cells with chemical or mechanical signals, makes traditional in vitro systems less effective to be used for modelling a biological system to mimic or simulate physiological activity. As a result, traditional in vitro systems have less predictive abilities and are therefore less useful to predict the functioning of in vivo systems.

It is known to combine a membrane-based cell culture insert with a perfusion device to enable the circulation of fluid through the cell culture, for instance to simulate shear stress from the fluid experienced by cells. In such a cell culture device, the cell culture chamber is open and is meant to be sealed by an insert. The insert may for instance mount a porous membrane or an impermeable surface on which the cells can be cultured. Such a system may use a cap to compress the insert on the microfluidic unit, keeping the fluidic layer sealed using direct plastic-on-plastic contact or a gasket. For the circulation of fluid, integrated peristaltic pumps can be used. A challenge with these known devices is to simulate the circulation of fluid in combination with high flow rates and on-chip unidirectional recirculation to further improve the simulation of physiological systems. Flow rates in the order of the millilitre per minute are difficult to achieve with pumping systems like embedded peristaltic pumps, which are typically one order of magnitude lower.

The present invention aims to provide an improved microfluidic cell culture device which at least partially accomplishes the aforementioned challenge.

To that end, a microfluidic cell culture device is provided according to claim 1. Specifically, a microfluidic cell culture device is provided comprising a body wherein said body comprises a fluid system comprising a first fluid chamber, a second fluid chamber, a cell culture chamber and a flow channel for allowing a flow of fluid from said first chamber, via said cell culture chamber, towards said second chamber, wherein the fluid system further comprises a return channel formed in said body allowing a flow of fluid from said second chamber to said first chamber.

For simulating the conditions for cell growth, fluid can flow via the flow channel from the first chamber via the cell culture chamber towards the second chamber, for instance under the influence of a pressure pump system coupled to the device. For instance, the pump system may be configured, and the device may be arranged to be coupled to such a pump system, to pressurise the flow channel when fluid is to flow from the first to the second chamber such that the fluid flows through the flow channel. As will be explained in greater detail below, the pressure pump, which may be a pneumatic pump, can be coupled to the first and/or the second chamber. Using a pressure pump, high flow rates can be achieved.

In a returning step, for instance different from the simulating step as mentioned above, fluid can flow from the second to the first chamber through the return channel Preferably no fluid, or substantially no fluid flows back through the flow channel A flow of fluid from said second chamber to said first chamber via the cell culture chamber is thus preferably prevented. The pump system may then further be configured, and the device may be arranged to be coupled to such a pump system, to pressurise the return channel when fluid is to flow from the second to the first chamber such that the fluid flows through the return channel Such a configuration is enabled by the fluid system comprising a return channel, wherein the return channel is different from the flow channel and does not flow through the cell culture chamber. The return channel being formed in the body enables on-chip (unidirectional) recirculation, while the ability to couple a pressure pump allows high flow rates. The fluid is preferably a liquid, for instance water based.

Accordingly, unidirectional closed loop recirculation is achieved by using the return channel to transfer the fluid from the second chamber back to the first chamber, without inverting the flow in the cell culture chamber. The closed loop recirculation allows on-chip storage of fluid and, therewith, enables the use of smaller volumes of fluid for an easier detection of secreted substances. After all, detection of a certain amount of substance is easier in a smaller volume of liquid, since the concentration of said substance is higher in the smaller volume.

The microfluidic cell culture device may be composed of a microfluidic network enabling pulsatile perfusion of fluids in a closed loop between the first and second fluid chambers, which may be integrated in or assembled on the body, and the cell culture chamber.

The actuation of the fluid may for instance be achieved by pressurisation of the first and/or second chambers or a direct pipetting of fluid via the first and/or second chambers. Such pressurisation is meant to enable peak values of flow rates in the order of the millilitre per minute, which is difficult to achieve with other integrated pumping systems like embedded peristaltic pumps.

Accordingly, a microfluidic cell culture device with on-chip recirculation that is re-sealable and compatible with high shear stress is provided. The microfluidic cell culture device allows for on-chip unidirectional closed loop recirculation of fluids stored on it, creating high shear stresses on, for example, the bottom side of a porous membrane on which the cells are provided, as will be explained in greater detail below. The combination of high flow rates and on-chip unidirectional recirculation enhance the performance of long-lasting cell culture experiments in physiologically relevant mechanical loading conditions, particularly in a setting that is compatible with a high throughput. The microfluidic cell culture device facilitates, in vitro, the representation of relevant features of in vivo systems while using standard formats and processes needed by industry. The microfluidic cell culture device therefore contributes to make medical research more predictive.

According to a preferred embodiment, the microfluidic cell culture device further comprises a fluid directing mechanism arranged for providing a substantially unidirectional flow in the flow channel. The fluid directing mechanism is aimed at directing a flow of fluid from the first chamber via the cell culture chamber to the second chamber through the flow channel and from the second chamber through the return channel to the first chamber. The fluid directing mechanism thereby aims to prevent a flow of fluid from the second chamber via the cell culture chamber to the first chamber through the flow channel, such that the flow of fluid in the flow channel, and thereby in cell culture chamber, is unidirectional. Generally, the flow direction in the flow channel equals that in the cell culture chamber. The fluid directing mechanism thereby does not aim to prevent the fluid to be at times stationary in the flow channel. The flow of fluid through the flow channel may be substantially unidirectional, such that the total amount of fluid flowing from the first chamber through the flow channel to the second chamber is greater than the total amount of fluid flowing from the second chamber through the flow channel to the first chamber. The fluid directing mechanism may be arranged in the flow channel. An additional fluid directing mechanism may be arranged in the return channel to provide a recirculating, unidirectional flow.

In a further embodiment, the fluid directing mechanism comprises at least one valve provided in at least one of the return channel and the flow channel. The valve can operate between an open state, in which the valve allows fluid to flow through the respective channel, and a closed state, in which the valve prevents fluid from flowing through the respective channel. If one valve is provided in the flow channel, the valve can be in the open state when the pump system pressurises the fluid for the fluid to flow from the first chamber to the second chamber in the simulating step as mentioned above, and in the closed state when the pump system pressurises the fluid to flow from the second to the first chamber such that, in the closed state of the valve, fluid flows through the return channel to prevent fluid from flowing from the second to the first chamber through the flow channel in the returning step as mentioned above. Alternatively, if the one valve is provided in the return channel, the fluid system may be configured such that, in the open state of the valve, fluid flows through the return channel when pressurised by the pump system, while in the closed state fluid flows through the flow channel. In this case, the valve is in the closed state when fluid is pressurised to flow from the first to the second chamber, and in the open state when fluid is pressurised to flow from the second to the first chamber to prevent the fluid from flowing from the second chamber through the flow channel to the first chamber.

Preferably, the valve is arranged in an upstream region of the respective channel. The upstream region may be the upstream half of the respective channel. For example, in case at least one valve is provided in the flow channel, the at least one valve is arranged upstream in the flow channel, i.e. towards the first fluid chamber as seen from the cell culture chamber. Placing the valve upstream may be advantageous by, at least to some extend, preventing exposure of the cell culture chamber to hydrostatic pressure needed to open the valve, for instance when a microfluidic check valve is used.

Alternatively, the valve could be arranged in a downstream region of the respective channel. The downstream region may be downstream half of the respective channel. For example, in case at least one valve is provided in the flow channel, the at least one valve is arranged downstream of the flow channel, i.e. towards the second fluid chamber as seen from the cell culture chamber. The valve being arranged downstream prevents fluid from entering the cell culture chamber from the side of the second fluid chamber when the fluid is pressurised by the pump system to flow from the second to the first chamber. Similarly, in case the at least one valve is provided in the return channel, the at least one valve may be arranged towards the first fluid chamber with respect to the second fluid chamber.

It is further preferred, that the flow channel and the return channel are provided with a valve. In such a configuration, the flow channel valve is preferably in the open state when the return channel valve is in the closed state to allow a flow of fluid through the flow channel and not the return channel, particularly when the fluid is pressurised by the pump system to flow from the first to the second chamber in the simulating step. It is further preferred if the flow channel valve is in the closed state when the return channel valve is in the open state to allow a flow of fluid through the return channel and not the flow channel, particularly when the fluid is urged to flow from the second to the first chamber in the returning step. Such a configuration enhances the unidirectional circulation of the fluid.

The one or more valves may comprise at least one pneumatic valve. A pneumatic valve has been found to close earlier and better. The pneumatic valve can be actuated via pneumatic control lines.

Preferably, the fluid directing mechanism comprises at least one one-way valve. The at least one one-way valve is then preferably provided in at least one of the return channel and the flow channel. The at least one one-way valve may be the at least one pneumatic valve. Preferably, the one-way valve however comprises a microfluidic passive check valve. The check valve has been found to be effective, as no external control of the check valve would be needed. Microfluidic passive check valves are as such known and may for instance comprise a membrane sealing an inlet for preventing back flow. An exemplary microfluidic passive check valve is disclosed in “integrated elastomeric components for autonomous regulation of sequential and oscillatory flow switching in microfluidic device” by Mosadegh, B., Kuo, C.-H., Tung, Y.-C., Torisawa, Y., Bersano-Begey, T., Tavana, H., and Takayama, S., as published in Nature Physics, 6(6), 433-437 (https://doi.org/10.1038/nphys1637).

In a further embodiment, at least one of the first and the second chambers are arranged to be coupled to a pump system for pressurising at least one of the first and the second chambers. It is preferred if the at least one of the first and second chambers is partially open for the pump system to pressurise the at least one of the first and second chambers. In other words, the first and/or second chamber is preferably formed as a hole in the body, which hole is closed at a bottom thereof, i.e. the hole is blind, and open on the top, i.e. it debouches at a surface of the body.

In case the pump system is coupled to the first chamber, the pump system can increase the pressure in the first chamber relative to the pressure in the second chamber to urge fluid to flow from the first to the second chamber. The pump system can similarly relatively decrease the pressure in the first chamber to urge fluid to flow from the second to the first chamber. Alternatively, in case the pump system is coupled to the second chamber, the pump system can increase the pressure in the second chamber relative to the pressure in the first chamber to urge fluid to flow from the second to the first chamber. The pump system can similarly relatively decrease the pressure in the second chamber to urge fluid to flow from the first to the second chamber. In case the pump system is coupled to both the first and the second chamber, the pump system can similarly manipulate the pressure difference between the first and the second chamber to urge fluid to flow from one chamber to another. According to a further aspect, a cell culturing system is provided comprising at least one microfluidic cell culture device and a pressure pump coupled to the body.

Preferably, the pump system is a pneumatic pressurisation system. The pneumatic pressurisation system can be actuated by a pneumatic actuation line for each connector coupling the pump system to the first chamber and/or the second chamber. As such, an alternating pressure difference between the first and second chamber may be applied via two pneumatic actuation line.

In a further embodiment, the cell culture chamber in the body is open towards a surface of the body for receiving a cell culture insert, wherein the cell culture insert and the cell culture chamber in the body together form a substantially closed area. The cell culture chamber may initially be open and may be meant to be sealed by such a cell culture insert with e.g. a basket shape, which may be composed of a rigid, cylindrical sidewall and a flat cell seeding area. The cell culture insert may for instance mount a porous membrane or an impermeable surface of gel interfaces. A cap may be used to compress the insert on the microfluidic cell culture device, keeping the fluidic layer sealed, for instance using direct plastic-on-plastic contact or a gasket. The insert-based device allows the cell culture chamber to be re-sealable.

The microfluidic cell culture device can be used for mechanical loading of cells in the bottom compartment of the insert using shear stress from the fluid flow as a stimulation, and compression of the cells in the upper chamber of the insert using pneumatic or hydraulic pressure, supplied by an external line or the pressure build-up in the flow channel. The flow channel connecting the first and second chambers and constituting the bottom of the cell culture chamber can be seeded with e.g. endothelial cells, to mimic vascularisation. The on-chip recirculation makes the microfluidic cell culture device compatible with e.g. immune cell recirculation.

In a further embodiment, the microfluidic cell culture device comprises a plurality of separated fluid systems. In one body, several cell cultures can be cultured separately from each other. Each of the fluid systems then has a respective first, second and cell culture chambers and may have a respective pressure pump coupled thereto. It may also be possible to couple one pressure pump to a plurality of fluid systems in a cell culturing system as mentioned above.

In a further embodiment, the body comprises a plurality of stacked layers, wherein the fluid system is formed by channels and openings formed in the plurality of layers. Such a configuration of the microfluidic cell culture device enables a precise and efficient manufacturing of the fluid system integrated in the body of the microfluidic cell culture device.

According to another aspect of the present invention, a method for culturing cells in a cell culture chamber is provided, comprising the steps of:
providing a microfluidic cell culture device as mentioned above, provided with cells in said cell culture chamber;
directing, in a first or simulating step, fluid from the first chamber through the flow channel and the cell culture chamber to the second chamber in a substantially unidirectional flow in said cell culture chamber; and
directing, in a second or returning step, the fluid from the second chamber through the return channel to the first channel, such that substantially no fluid is directed from the second chamber to the first chamber via the flow channel.

The method allows the unidirectional flow of fluid through the fluid system of the microfluidic cell culture device, in particular in the cell culture chamber, for the fluid flow to provide a sufficient shear stress to the cells to sufficiently mimic a physiologically relevant condition, e.g. interstitial flow through bone cells, to enhance the predictability of medical research when applied to an in vivo system.

Preferably, the step of directing fluid from the first chamber to the second chamber further comprises opening any valve, see above, in the flow channel and/or closing any valve in the return channel.

It is further preferred, if in the step of directing fluid from the second chamber to the first chamber further comprises closing any valve in the flow channel and/or opening any valve in the return channel.

The present invention is hereafter further elucidated with reference to the attached drawings, wherein:

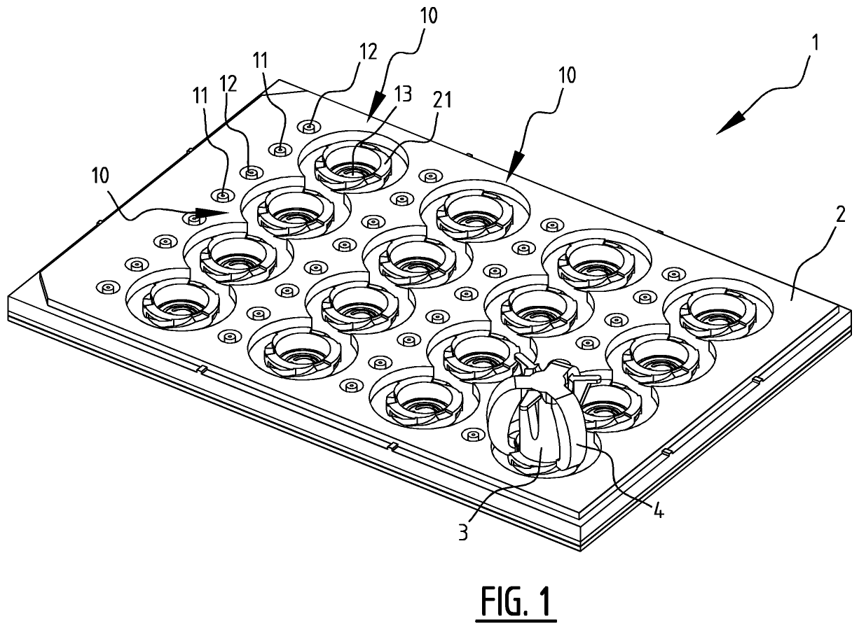

FIG. 1 shows an example of the microfluidic cell culture device;

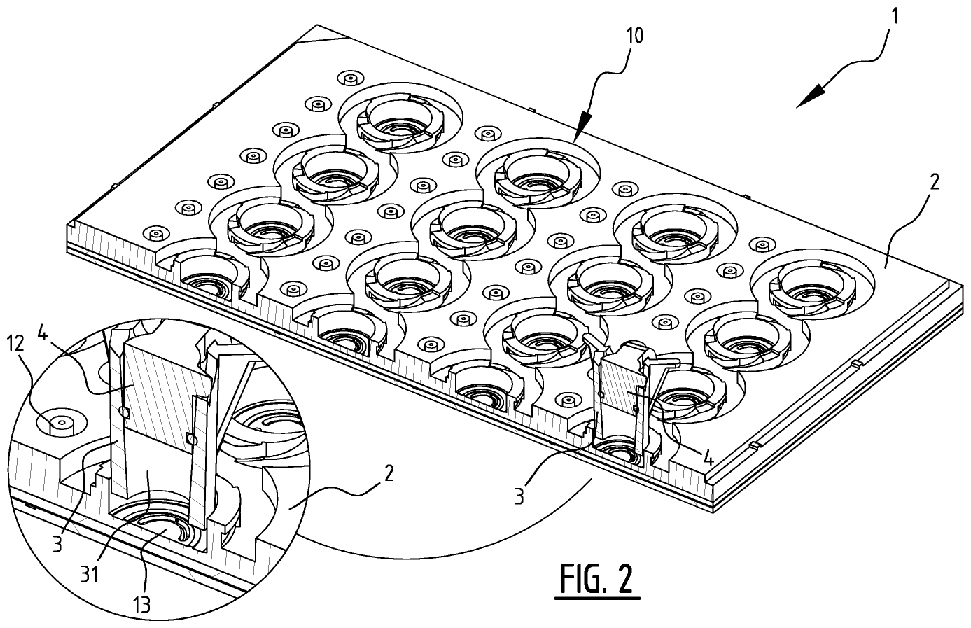

FIG. 2 shows a cross section of a cell culture insert received by the cell culture chamber;

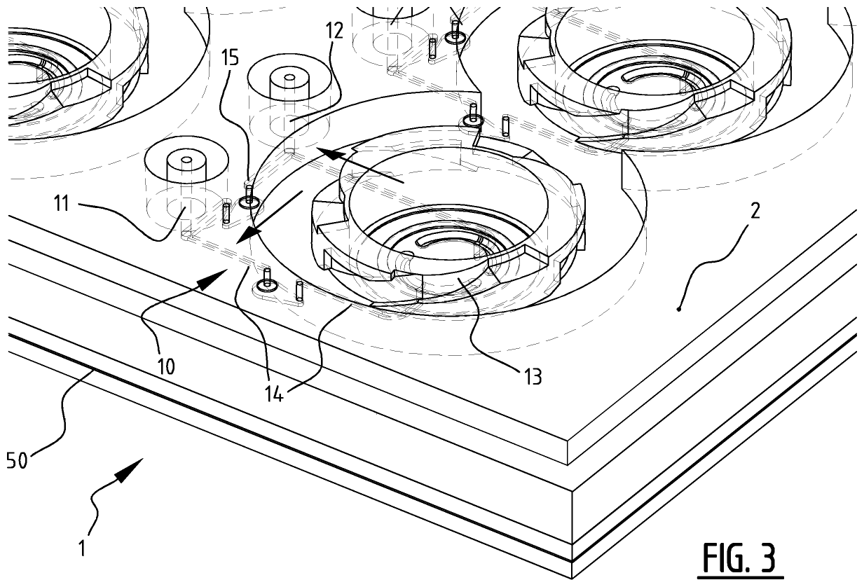

FIG. 3 shows a more detailed view of a fluid system in a close up view of the microfluidic cell culture device;

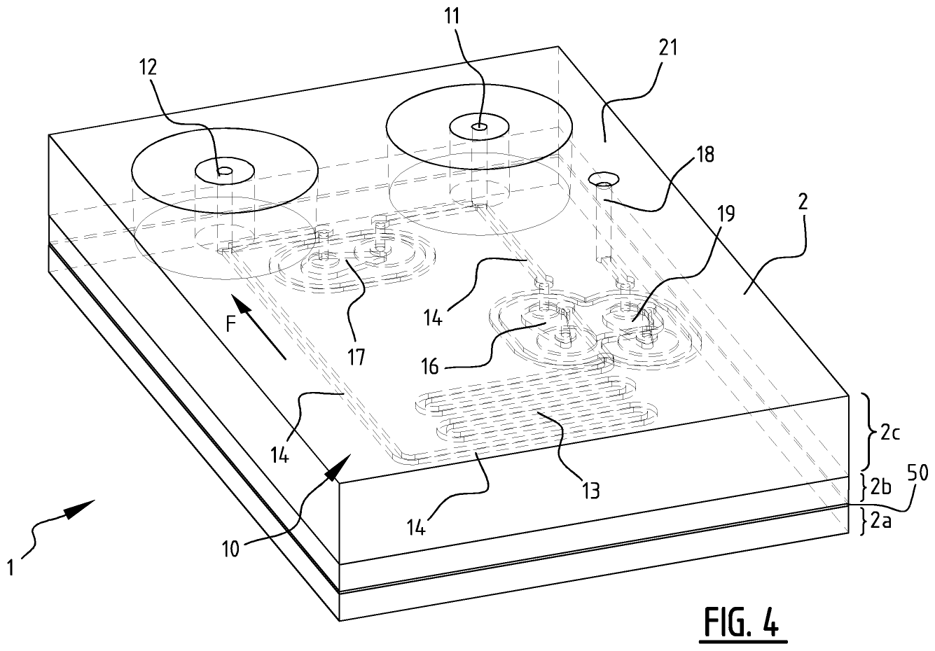

FIG. 4 shows another example of the microfluidic cell culture device;

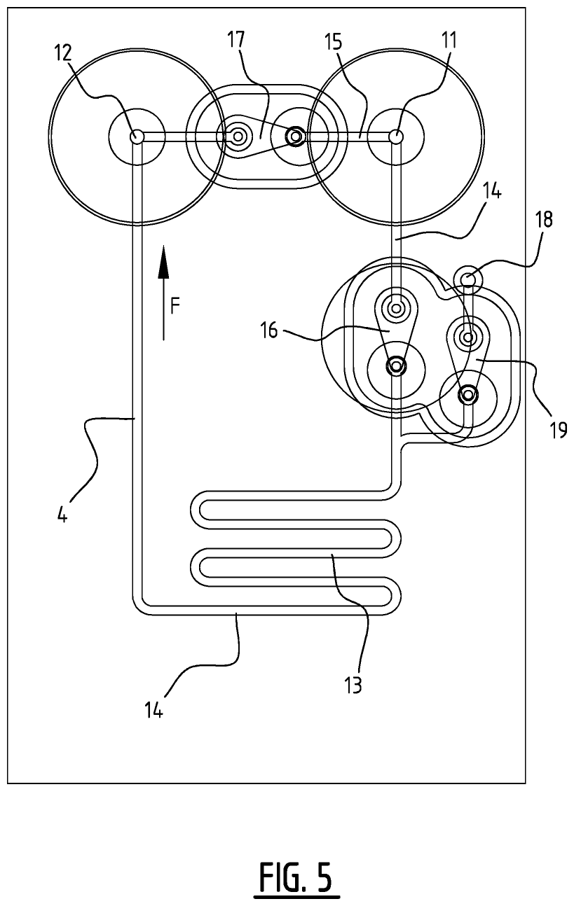

FIG. 5 shows a top view of the microfluidic cell culture device in FIG. 4;

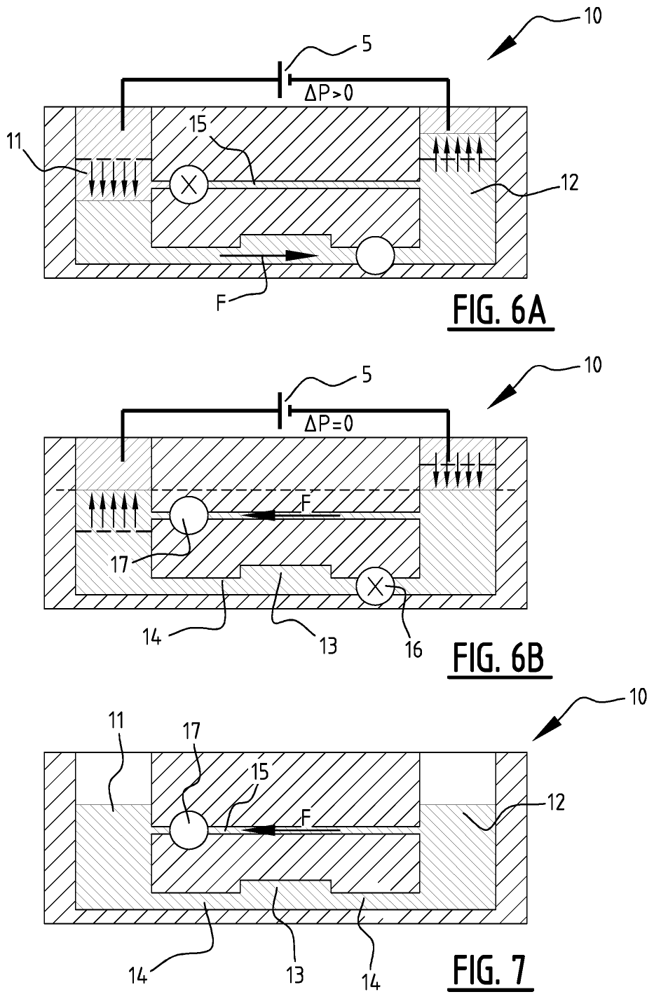

FIGS. 6A and 6B show the working principle of an example of the fluid system of the microfluidic cell culture device; and

FIG. 7 shows another example of the fluid system.

In FIG. 1 an embodiment of the microfluidic cell culture device 1 is shown. The microfluidic cell culture device 1 comprises a body 2. The body 2 comprises a plurality of separated fluid systems Each fluid system 10 comprises a first fluid chamber 11, a second fluid chamber 12 and a cell culture chamber 13. The cell culture chamber 13 in the body 2 is open towards a surface 21 of the body 2 for receiving a cell culture insert 3. A locking cap 4 can be used to compress the cell culture insert 3 onto the fluid system 10.

FIG. 2 shows that the cell culture insert 3 and the cell culture chamber 13 in the body 2 together form a substantially closed area 31. The cell culture chamber 13 is configured to be sealed by the cell culture insert 3 having a substantially cylindrical sidewall and a flat cell seeding area. In particular, the side walls of the cell culture insert 3 may be slightly skewed by approximately 3 degrees. As such, the cell culturing insert 3 could be described as having a frustoconical shape, which herein is regarded as substantially cylindrical for reasons of simplicity.

FIG. 3 shows a close up view of a fluid system 10 comprising a first fluid chamber 11, a second fluid chamber 12, a cell culture chamber 13 and a flow channel 14 for allowing a flow of fluid from said first chamber 11, via said cell culture chamber 13, towards said second chamber 12 upon coupling of a pressure pump system to said fluid system 10. The fluid system 10 further comprises a return channel 15 formed in said body 2 allowing a flow of fluid from said second chamber 12 to said first chamber 11.

In FIGS. 4 and 5 another embodiment of the microfluidic cell culture device 1 is shown and FIG. 5 shows a top view of this embodiment. The microfluidic cell culture device 1 comprises a body 2. The body 2 comprises a single fluid system 10. The fluid system 10 comprises a first fluid chamber 11, a second fluid chamber 12, a cell culture chamber 13 and a flow channel 14 for allowing a flow of fluid from said first chamber 11, via said cell culture chamber 13, towards said second chamber 12 upon coupling of a pressure pump system to said fluid system 10. The fluid system 10 further comprises a return channel 15 formed in said body 2 allowing a flow of fluid from said second chamber 12 to said first chamber 11. The microfluidic cell culture device 1 further comprises a flow channel valve 16 provided in the flow channel 14 and a return channel valve 17 provided in the return channel 15. The flow channel valve 16 and the return channel valve 17 are arranged for providing a substantially unidirectional flow with a single flow direction F in the flow channel 14. The flow channel valve 16 is arranged in a upstream region of the flow channel 14. The body 2 comprises a plurality of stacked layers comprising a first layer 2a, a second layer 2b and a third layer 2c, wherein the fluid system 10 is formed by channels and openings formed in the plurality of layers. A membrane layer 50 is interposed between the first and second layers 2a, 2b. The valves 16, 17 are formed in the first layer 2a, second layer 2b and the interposing membrane 50. The flow channel 14 and the return channel 15 are formed in the second layer 2b. The first chamber 11 and the second chamber 12 are formed in the third layer 2c. The microfluidic cell culture device further comprises an opening 18 for manipulating the fluid in the fluid system Through the opening 18 fluid, and in particular a suspension of cells, can be added to the fluid system 10 and the fluid mixture in the fluid system 10 can be adjusted via the opening 18. The opening 18 is provided in the body 2, particularly in the surface 21 of the body 2. The opening 18 forms a passage to the flow channel 14. The microfluidic cell culture device 1 comprises an opening valve 19 provided between the opening 18 and the flow channel 14 to separate the fluid system 10 from the opening 18 when closing the opening valve 19. In the shown chip, the opening valve 19 is a one-way valve allowing only flow from the opening 18 into the chip. The opening 18, in cooperation with the opening valve 19, allows bringing fluid, such as the aforementioned suspension of cells, directly into the chip near the cell culture chamber, thereby bypassing the first 11 and/or second fluid chamber 12.

FIGS. 6A and 6B are a schematic representation of the working principle of an embodiment of the fluid system 10. As shown in FIG. 6A, the first chamber 11 and the second chamber 12 are arranged to be coupled to a pump system 5 for pressurising the first chamber 11 and the second chamber 12. The first chamber 11 and the second chamber 12 are partially open for the pump system 5 to pressurise the first chamber 11 and the second chamber 12. The flow channel 14 is provided with a flow channel valve 16 and the return channel 15 is provided with a return channel valve 17. In this configuration, the flow channel valve 16 is in the open state when the return channel valve 17 is in the closed state to allow a flow of fluid through the flow channel 14 and not the return channel 15, according to the single flow direction F, particularly when the fluid is pressurised by the pump system 5 to flow from the first chamber 11 to the second chamber 12. Because the pump system 5 is coupled to the first chamber 11 and the second chamber 12, the pump system 5 can manipulate the pressure difference ΔP between the first chamber 11 and the second chamber 12 to urge fluid to flow from one chamber to another. By being coupled to the first chamber 11, the pump system 5 can increase the pressure in the first chamber 11 relative to the pressure in the second chamber 12 to urge fluid to flow from the first chamber 11 to the second chamber 12. The pump system 5 can similarly decrease the pressure in the second chamber 12 to urge fluid to flow from the first chamber 11 to the second chamber 12.

As shown in FIG. 6B, The flow channel valve 16 is in the closed state when the return channel valve 17 is in the open state to allow a flow of fluid through the return channel 15 and not the flow channel 14, according to the single flow direction F, particularly when the fluid is urged to flow from the second chamber 12 to the first chamber 11. Such a configuration enhances the unidirectional circulation of the fluid. By being coupled to the second chamber 12, the pump system 5 can increase the pressure in the second chamber 12 relative to the pressure in the first chamber 11 to urge fluid to flow from the second chamber 12 to the first chamber 11. The pump system 5 can similarly decrease the pressure in the first chamber 11 to urge fluid to flow from the second chamber 12 to the first chamber 11. It is noted that in stead of downstream, the valve 16 could have been provided upstream of the cell culture area 13.

FIG. 7 shows another example of the fluid system 10, wherein the flow channel 14 provides a flow resistance to a flow of fluid that is significantly larger than the flow resistance provided by the return channel 15, such that the flow of fluid from the second chamber 12 to the first chamber 11 through the flow channel 14 is insignificant in comparison with the flow of fluid through the return channel 15 when the fluid is urged to flow from the second chamber 12 to the first chamber 11 and the return channel valve 17 is open. If the return channel valve 17 is closed, the fluid can be directed to flow through the flow channel 14.

The above description of the attached drawings is provided merely for illustrative purposes to contribute to comprehension of the present invention, and is not intended to limit the scope of the appended claims in any way or form.

1. A microfluidic cell culture device comprising a body wherein said body comprises a fluid system comprising a first fluid chamber, a second fluid chamber, a cell culture chamber and a flow channel for allowing a flow of fluid from said first chamber, via said cell culture chamber, towards said second chamber upon coupling of a pressure pump system to said fluid system, wherein the fluid system further comprises a return channel formed in said body allowing a flow of fluid from said second chamber to said first chamber.

2. The microfluidic cell culture device according to claim 1, further comprising a fluid directing mechanism arranged for providing a substantially unidirectional flow in the flow channel.

3. The microfluidic cell culture device according to claim 2, wherein the fluid directing mechanism comprises at least one valve provided in at least one of the return channel and the flow channel.

4. The microfluidic cell culture device according to claim 3, wherein the fluid directing mechanism comprises at least one pneumatic valve.

5. The microfluidic cell culture device according to claim 3, wherein the fluid directing mechanism comprises at least one one-way valve.

6. The microfluidic cell culture device according to claim 5, wherein the one-way valve comprises a microfluidic passive check valve.

7. The microfluidic cell culture device according to claim 3, wherein the valve is arranged in a upstream region of the respective channel.

8. The microfluidic cell culture device according to claim 3, wherein both the flow channel and the return channel are provided with a valve.

9. The microfluidic cell culture device according to claim 1, wherein at least one of the first and the second chambers are arranged to be coupled to a pump system for pressurizing at least one of the first and the second chambers.

10. The microfluidic cell culture device according to claim 9, wherein the pump system is a pneumatic pressurization system.

11. The microfluidic cell culture device according to claim 1, wherein the cell culture chamber in the body is open towards a surface of the body for receiving a cell culture insert, wherein the cell culture insert and the cell culture chamber in the body together form a substantially closed area.

12. The microfluidic cell culture device according to claim 1, comprising a plurality of separated fluid systems.

13. The microfluidic cell culture device according to claim 1, wherein the body comprises a plurality of stacked layers, wherein the fluid system is formed by channels and openings formed in the plurality of layers.

14. A method for culturing cells in a cell culture chamber, comprising the steps of:
providing a microfluidic cell culture device according to claim 1, provided with cells in said cell culture chamber;
directing, in a first step, fluid from the first chamber through the flow channel and the cell culture chamber to the second chamber in a substantially unidirectional flow in said cell culture chamber, and;
directing, in a second step, the fluid from the second chamber through the return channel to the first chamber, such that substantially no fluid is directed from the second chamber to the first chamber via the flow channel.

15. The method according to claim 14, wherein the step of directing fluid from the first chamber to the second chamber further comprises closing each valve in the return channel and opening each valve in the flow channel, wherein the step of directing fluid from the second chamber to the first chamber further comprises closing each valve in the flow channel and opening each valve in the return channel.
